(19) United States
(12) Patent Application Publication
(10) Pub. No.: US 2024/0344010 A1
(43) Pub. Date: Oct. 17, 2024

(54) TEMPERATURE CONTROLLED BIOLOGICAL GROWTH SURFACES FOR HIGHER MICROBIAL ACTIVITY IN BIOPROCESSING

(71) Applicant: University of Washington, Seattle, WA (US)

(72) Inventors: Raymond RedCorn, Seattle, WA (US); Bruce J. Godfrey, Seattle, WA (US); Mari-Karoliina Henriikka Winkler, Seattle, WA (US); Samuel Bryson, Seattle, WA (US)

(73) Assignee: University of Washington, Seattle, WA (US)

(21) Appl. No.: 18/293,011

(22) PCT Filed: Aug. 11, 2022
(86) PCT No.: PCT/US2022/074816

§ 371 Date: Jan. 29, 2024

Related U.S. Application Data
(60) Provisional application No. 63/233,071, filed on Aug. 13, 2021.

(51) Int. Cl. C12M 1/34; C02F 3/10; C02F 3/30; C02F 3/34; C02F 101/16; C12M 1/12

(52) U.S. Cl. CPC C12M 41/12; C02F 3/102; C02F 3/104; C02F 3/108; C02F 3/307; C02F 3/341; C12M 25/02; C02F 2101/16; C02F 2101/166

(57) ABSTRACT

A temperature controlled biological growth surface, including a biomass growth surface configured to support a biomass, and a temperature source in thermal communication with the biomass growth surface. Further, a method for removing one or more of nitrogen, carbon, or phosphate from a medium, including contacting a temperature controlled biological growth surface with the medium, where the biological growth surface comprises an organism configured to remove the one or more of nitrogen, carbon, or phosphate from the medium when within a temperature range, and heating the temperature controlled biological growth surface to within the temperature range in order to allow the organism to remove the one or more of nitrogen, carbon, or phosphate from the medium, or cooling the temperature controlled biological growth surface to within the temperature range in order to allow the organism to remove the one or more of nitrogen, carbon, or phosphate from the medium.

### CROSS-REFERENCE TO RELATED APPLICATION

This application claims the benefit of U.S. Provisional Application No. 63/233,071 filed Aug. 13, 2021, the entire disclosure of which is hereby incorporated by reference.

### BACKGROUND

Conventional nitrogen and carbon removal from high nitrogen and carbon substances, through processes such as nitrification and heterotrophic denitrification to produce nitrogen gas (N₂), results in significant sludge and CO₂ production, and requires high aeration energy. In comparison, anaerobic ammonium oxidizing (anammox) bacteria produce only a quarter of the excess sludge, produces less CO₂, and reduces the energy required for aeration by 60%. Anammox bacteria have a unique metabolic ability to combine ammonium and nitrite to produce nitrogen gas (N₂) in the absence of oxygen. Anammox bacteria are autotrophic, and thus carry out this process without a need for an organic carbon source. Anammox bacterial metabolism of ammonium and nitrite to produce nitrogen gas (N₂) provides new possibilities for biological nitrogen removal from wastewater. The application of anammox bacteria to the treatment of high-temperature wastewater has been demonstrated to offer substantial reduction in energy demand for nitrogen removal from wastewater. Although this has led to interest in applying anammox bacteria to mainstream wastewater treatment, a prohibitive challenge is that anammox bacterial activity sharply decreases at the lower temperatures common in the mainstream treatment of wastewater. Similarly, other biological processes like anaerobic digestion of waste, conversion of carbon substrates into biogas, and fermentation of brewer's wort into ethanol by yeast, can benefit from keeping the active organisms at a warmer temperature. Altering the temperature of large volumes of water, such as wastewater, is economically prohibitive due to the significant energy required and its associated costs.

The use of microbes in biofilms attached to gas-permeable membranes is one of the most efficient ways to operate a bioprocess which relies on the feed of a gas such as oxygen, or methane, or carbon dioxide as it can meter the gas delivery very precisely and virtually eliminate waste. The addition of growth surface temperature control to this type of bioreactor generally offers further improved economy and efficiency for many other types of bioprocesses besides the treatment of wastewater streams.

A need therefore exists for controlling the temperature of organism(s) performing the biological process, without the high energy cost of controlling the temperature of large volumes of water.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a temperature controlled biological growth surface, including a biomass growth surface configured to support a biomass; and a temperature source in thermal communication with the biomass growth surface is disclosed.

In another aspect, a method for removing one or more of nitrogen, carbon, or phosphate from a medium, including contacting a temperature controlled biological growth surface with the medium, wherein the biological growth surface comprises an organism configured to remove the one or more of nitrogen, carbon, or phosphate from the medium when within a temperature range, heating the temperature controlled biological growth surface to within the temperature range in order to allow the organism to remove the one or more of nitrogen, carbon, or phosphate from the medium, or cooling the temperature controlled biological growth surface to within the temperature range in order to allow the organism to remove the one or more of nitrogen, carbon, or phosphate from the medium is disclosed.

In yet another aspect, a method for converting one or more of nitrogen, carbon, or phosphate from a medium, including contacting a temperature controlled biological growth surface with the medium, wherein the biological growth surface comprises an organism configured to remove the one or more of nitrogen, carbon, or phosphate from the medium when within a temperature range, heating the temperature controlled biological growth surface to within the temperature range in order to allow the organism to convert the one or more of nitrogen, carbon, or phosphate from the medium, or cooling the temperature controlled biological growth surface to within the temperature range in order to allow the organism to convert the one or more of nitrogen, carbon, or phosphate from the medium is disclosed.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A shows a perspective view diagram for heating of biomass using resistance heating with a wire (or carbon fiber) on a flat membrane, in accordance with the present technology;

FIG. 1B shows a cross-sectional view for direct heating of biomass using resistance heating with a wire on a flat membrane, in accordance with the present technology;

FIG. 2A shows heating of a biomass through resistance heating of the interior surface of a cylindrical growth surface membrane, in accordance with the present technology;

FIG. 2B shows heating of a biomass on the exterior of a membrane through resistance heating by a carbon fiber integrated into the membrane, in accordance with the present technology;

FIG. 2C shows heating of a biomass on the exterior of a membrane through resistance heating by a carbon fiber woven into the membrane, in accordance with the present technology;

FIG. 3 shows resistance heating of a hydrogel comprising carbon fiber embedded within the hydrogel, and an optional membrane support surface, in accordance with the present technology;

FIG. 4 shows direct resistance heating of biomass grown directly on a carbon fiber resistance heating element, in accordance with the present technology;

FIG. 5 shows a schematic for gas convection with the biomass growth on one membrane surface and the temperature-controlled gas in communication with the other membrane surface, in accordance with the present technology;

FIG. 6 shows a schematic for circulation of a temperature-controlled gas with the biomass growth on one membrane surface and the liquid in communication with the other membrane surface, in accordance with the present technology;

FIG. 7A shows a diagram for direct heating by radiation heat of hydrogel beads in contact with a biomass, in accordance with the present technology;

FIG. 7B shows an example biomass particle 1000, in accordance with the present technology;

FIG. 8 shows a diagram of a hydrogel comprising a biomass supported by a membrane such as a gas exchange membrane, and with heating of the hydrogel by solar radiation and incorporation of a radiation filter, in accordance with the present technology;

FIG. 9 shows a diagram of solar radiation in communication with a membrane biomass growth surface, a selective radiation-blocking material, and a hydrogel matrix comprising the biomass in contact with the other membrane surface of the biomass growth surface, in accordance with the present technology;

FIG. 10 shows a diagram of a reactor having a biomass growth surface, in accordance with the present technology;

FIG. 11 is a graph showing the nitrogen removal capacity of ammoxic bacteria with and without carbon black, in accordance with the present technology;

FIG. 12A is a graph showing the removal efficiency of total inorganic nitrogen (TIN), in accordance with the present technology;

FIG. 12B is a graph showing the removal of various nitrogen species over time, in accordance with the present technology;

FIG. 13 shows three microscope images of biomass and carbon powder suspended in a biomass support, in accordance with the present technology;

FIG. 14A is a graph showing the removal efficiency for TIN, in accordance with the present technology; and

FIG. 14B is a graph showing the removal of various nitrogen species over time, in accordance with the present technology.

### DETAILED DESCRIPTION

While illustrative embodiments have been illustrated and described, it will be appreciated that various changes can be made therein without departing from the spirit and scope of the disclosure.

The present disclosure provides a device which selectively heats or cools biological organisms in contact with nitrogen, carbon and or phosphate-containing substances, and methods for use of the device, to metabolize nitrogen, carbon and or phosphate from the substances, reducing the undesirable by-products and prohibitive costs associated with current nitrogen and carbon-removal or harvesting treatment methods as well as fermentation technology.

The device comprises a heated or cooled biological growth surface, which selectively heats or cools a biological organism or consortium of organisms', thus eliminating the high energy requirement and associated costs of heating or cooling large volumes of substances. Use of bacteria such as anaerobic ammonium oxidizing (anammox) bacteria to effect nitrogen removal from substances significantly reduces sludge by-product, CO₂ production, and aeration energy required by conventional nitrification and heterotrophic denitrification processes.

In addition to anammox bacteria, other biomass that prefer warmer temperatures, such as anaerobic bacteria, nitrifying bacteria, methanogen or methanotroph, denitrifying organisms, sulfate reducers, sulfate oxidizers, carbon- or nitrogen-processing heterotrophic or phototrophic prokaryotes, carbon- or nitrogen-processing heterotrophic or phototrophic eukaryotes, fungal strains such as yeast, and combinations thereof, can be used. Some of these organisms can be found in anaerobic digesters or fermenters utilized in the food and biochemical industry. Other disclosed organisms can be found in environments in which carbon feed stock is used as fuel, such as in petrochemical, pharmaceutical, and food production applications.

Use of the device described herein to selectively heat or cool biological organisms, such as biomass, bacteria, biofilm, yeast, and other biological organisms, then contacting the heated or cooled biological organisms with nitrogen, carbon, and/or phosphate-containing substances effects nitrogen, carbon, and phosphate removal or harvesting from the substance. Embodiments in the present disclosure are not limited to aqueous applications, but can be applied to other liquids, as well as solid or gas mediums. The nitrogen-, carbon-, and/or phosphate-containing substances of the disclosure can be mediums such as liquid, solid, and/or gas. A liquid medium comprising a nitrogen-, carbon-, and/or phosphate-containing substance can comprise wastewater, contaminated water, water comprising a nitrogen-, carbon-, and/or phosphate-containing substance, a carbon feed stock used for fuel, a carbon feed stock for pharmaceutical production, and/or a carbon feed stock for food production. A gas medium can comprise ambient air comprising a nitrogen-, carbon-, and/or phosphate-containing substance, a gas mixture comprising a nitrogen-, carbon-, and/or phosphate-containing substance, or a waste gas. A solid medium comprising a nitrogen-, carbon-, and/or phosphate-containing substance could comprise a soil sample or contaminated rock.

The primary means for heating or cooling biological organisms utilized by the device and the methods described herein comprises electric resistance heating; convection heating and cooling by a heated or cooled gas or liquid; and/or radiation heating. Such heating or cooling can heat or cool the bacteria, biomass, biofilm, yeast, or other biological organisms (hereinafter referred to as biomass) directly; through a matrix comprising the biomass, such as a hydrogel or other porous matrix configured to permit diffusion of growth substrates and metabolic products in and out of the matrix; or a biomass growth surface upon which the biological organisms, biomass, biofilm, or matrix comprising the biomass is deposited or grown.

Certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination. Accordingly, both devices and methods of using the devices are disclosed.

In some embodiments, the disclosure provides a biomass growth surface heated or cooled with resistive heat, gas convection, liquid convection, radiation heat, and combinations thereof. In certain embodiments, the heated or cooled biological growth surface comprises a biofilm on the biomass growth surface. In other embodiments, the heated or cooled biological growth surface or biomass growth surface comprises a hydrogel in contact with a biomass. Embodiments comprising a hydrogel can comprise a hydrogel layer; a plurality of beads comprising a hydrogel matrix; or other hydrogel-containing configuration or composition. Hydrogel beads can comprise hydrogel that is smooth, regular, irregular, rough, porous. In certain embodiments, hydrogel beads comprise biomass, particles, pigment, other additives, or combinations thereof.

In other embodiments, the heated or cooled biological growth surface or biomass growth surface comprises a porous matrix in contact with a biomass. Such porous matrix is configured to permit diffusion of growth substrates and metabolic products in and out of the matrix. Embodiments comprising a porous matrix can comprise a porous matrix layer; a plurality of beads comprising a porous matrix; or other porous matrix-containing configuration or composition. Porous matrix beads can be smooth, regular, irregular, rough, porous. In certain embodiments, porous matrix beads comprise biomass, particles, pigment, other additives, or combinations thereof.

In the foregoing and the following embodiments disclosed herein, biomass can comprise a biological organism, or a consortium of organisms wherein more than one type of organism is utilized. In such embodiments, the biological organism or consortium of organisms can comprise bacteria, biomass, biofilm, biomass in contact with a matrix such as a hydrogel or other porous matrix, anaerobic ammonium oxidizing (anammox) bacteria, nitrifying bacteria, methanogen or methanotroph, denitrifying organisms, sulfate reducers, sulfate oxidizers, carbon- or nitrogen-processing heterotrophic or phototrophic prokaryotes, carbon- or nitrogen-processing heterotrophic or phototrophic eukaryotes, fungal strains such as yeast, and combinations thereof.

In some embodiments, the biological growth surface comprises a membrane, a carbon fiber filament, a carbon fiber ribbon, a metal plate, a pigmented membrane, or a combination thereof.

In some embodiment, a biomass growth surface heated with electric resistive heat or resistance-based heating elements is disclosed. In certain embodiments, a heating wire can be located inside a hollow fiber membrane, or it can be located on the surface of a membrane. A low voltage (e.g., 24V, powering parallel circuits, each 5 ft segments of 36-gauge nichrome) can then be applied to prevent the system from overheating the biomass. The heating wire can comprise nichrome. The electric circuit or nichrome circuit can be controlled by a PID (proportional-integral-derivative) temperature controller which controls the temperature of the biomass growth surface, and a temperature sensor (e.g., infrared sensor, thermocouple, or RTD (“Resistance Temperature Detector”) which detects the temperature of the biomass growth surface. For example, the setpoint temperature range for a biological organism such as anammox could be between about 20° C. to about 60° C., from about 20° C. to about 50° C., from about 25° C. to about 45° C., or from about 30° C. to about 40° C. Such a temperature range permits a biological organism such as anammox, aerobic thermophilic organisms, and/or anaerobic thermophilic organisms to achieve their maximal growth rate, and consequently achieve high nitrogen-, carbon-, and/or phosphate removal or harvesting efficiencies.

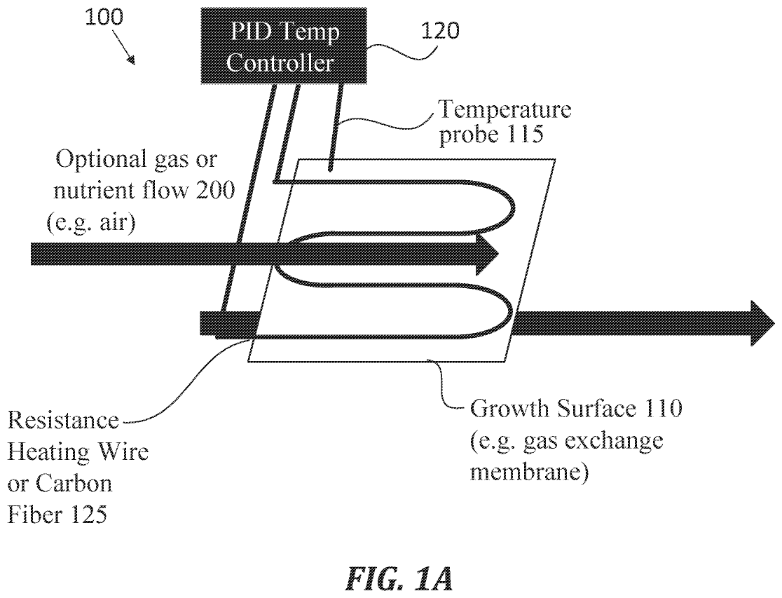

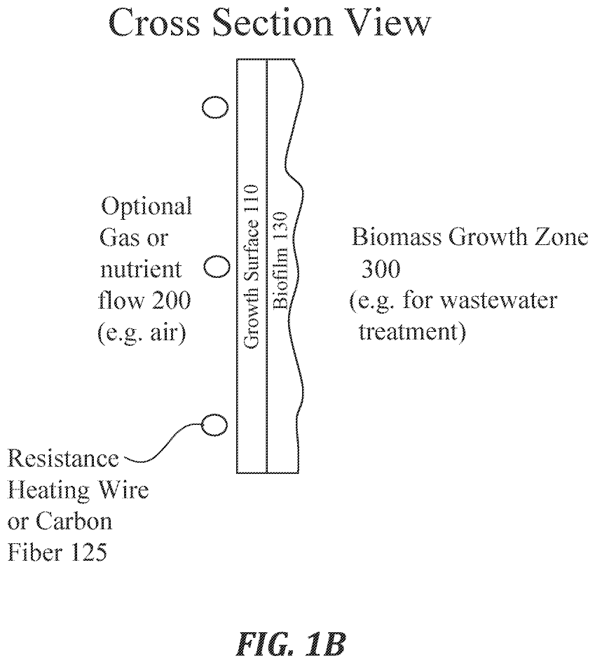

FIG. 1A shows a perspective view of a system 100 for temperature controlling a flat biomass growth surface 110 wherein the biomass growth surface 110 is, for example, a gas exchange membrane. In some embodiments, the growth surface 110 is flat, but it may be selected from a form factor selected from substantially planar, substantially cylindrical, and a plurality of beads, as described herein. In some embodiments, the growth surface 110 is a gas exchange membrane. In some embodiments, the growth surface is a ceramic membrane, a metallic membrane, a hollow-fiber membrane, or a combination thereof. In some embodiments, the biological growth surface is a carbon fiber filament, a carbon fiber ribbon, a metal plate or tube, a metallic surface modified to accept a biofilm or hydrogel coating, a pigmented membrane, or a combination thereof.

In some embodiments, the temperature is controlled through a PID temperature controller 120. The PID temperature controller may be coupled, either physically or communicatively, to a temperature probe 115 and a temperature source 125. As illustrated in FIG. 1A, the temperature source 125 may be resistance heating wire or carbon wire. In some embodiments, the temperature source 125 may be a combination of resistance heating wire or carbon wire. In some embodiments, the temperature source 125 is a heat source selected from resistive heat, gas convection heat, liquid convention heat, radiation heat, heat from combustion, heat from friction, and combinations thereof, as described herein. In some embodiments, a gas or nutrient flow 200 passes over one side of the growth surface 110. In some embodiments, the gas or nutrient flow 200 is air.

FIG. 1B shows the cross-section view of a system (such as system 100) for heating a flat biomass growth surface 110 wherein the biomass growth surface 110 is a gas exchange membrane, in accordance with the present technology. In some embodiments, the growth surface 110 supports a biofilm 130 on the side opposite the resistance temperature source 125. In some embodiments, gas or nutrient flow 200 contact the growth surface 110 on one side. In some embodiments, on the side opposite the gas or nutrient flow 200 is a biomass growth zone 300. In some embodiments, the biomass growth zone 300 is located on the side opposite the temperature source 125.

In operation, as shown in FIGS. 1A and 1B the temperature source 125, illustrated as a resistance heating wire provides heat to the biofilm 130. The biofilm 130 may then used for treating a wastewater stream in the biomass growth zone 300.

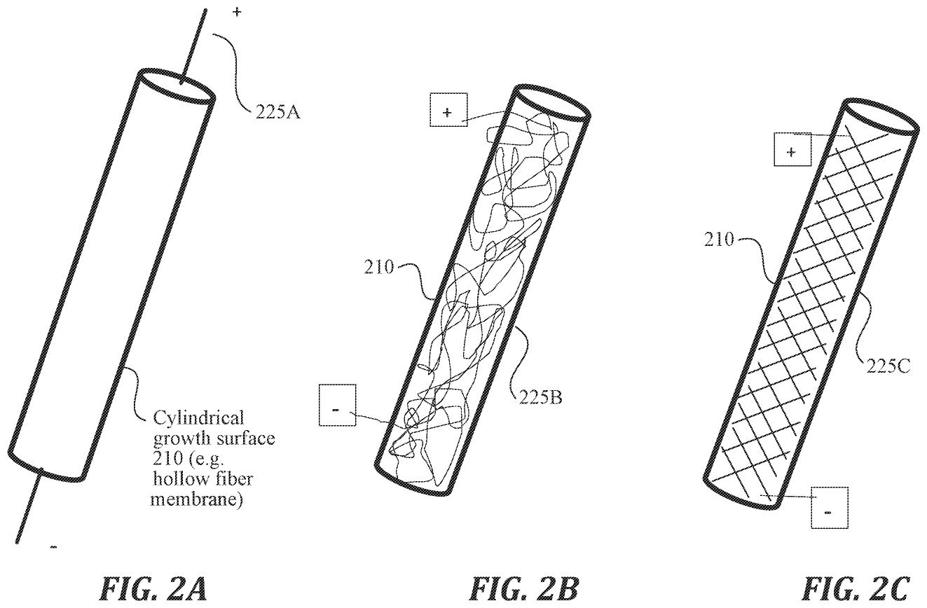

FIG. 2A shows temperature controlling of a cylindrical surface 210 including a temperature source 225A, in accordance with the present technology. In some embodiments, the temperature source 225A is a resistance heating wire, a carbon fiber, or a nichrome wire is located through a hollow fiber membrane. In operation, the resistance heating wire 225A may be supplied with a given voltage, and the biomass (not shown) is placed or grown on the outside surface of the membrane.

In some embodiments, the resistance heating element is integrated into a flat, cylindrical, or other shaped membrane (e.g., woven in), for example, during the membrane manufacturing process. In a further embodiment, carbon fibers, or another heating element, could be used to strengthen the membrane while concurrently providing heat when a voltage is applied to the carbon fiber or other heating element.

FIG. 2B shows heating of a cylindrical surface 210 including a temperature source 225B, in the form of a carbon fiber integrated into the membrane 210 and a voltage applied to the carbon fiber 225B. The carbon fiber 225B provides both strength to the growth surface and heating to the biomass. In some embodiments, the carbon fiber 225B may be integrated into a flat surface.

In some embodiments, carbon fiber 225B, or another temperature source 225, is integrated into the biological growth surface 210. In some embodiments, the biological growth surface 210 includes a hydrogel or other porous matrix in contact with the biomass. In a further embodiment, an underlying surface (e.g., a membrane) may add additional support to the biological growth surface 210, or the hydrogel or other porous matrix may be engineered and reinforced for adequate strength to sustain biomass without external structural support.

FIG. 2C shows temperature controlling of a biomass on the exterior of a membrane through resistance heating by a temperature source 225C in the form of carbon fiber woven into the membrane, in accordance with the present technology. In some embodiments, carbon fiber is woven into the growth surface 210. While growth surface 210 is illustrated as a cylinder, it should be understood that the growth surface 210 could also be flat or shaped as a plurality of beads as described herein.

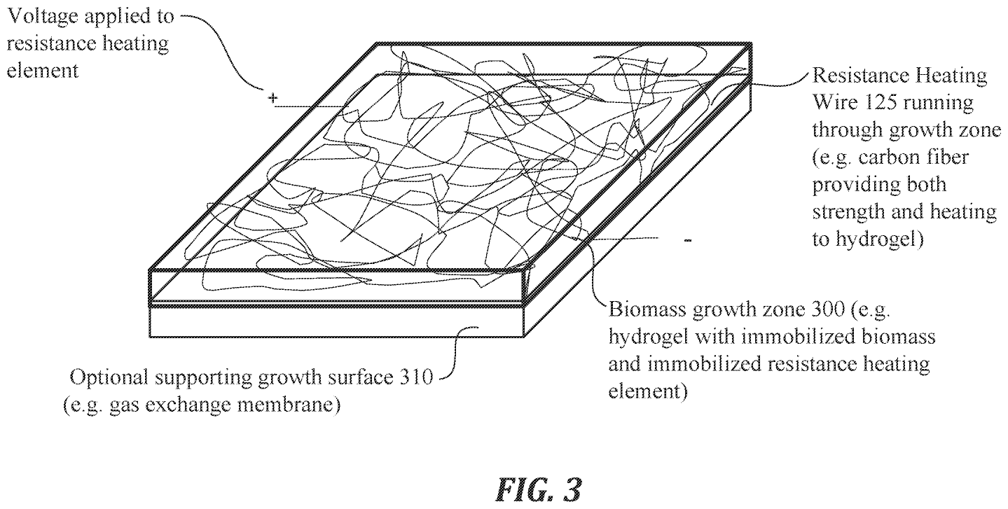

FIG. 3 shows direct heating of a flat surface hydrogel 300 in contact with a biomass, wherein the temperature source 125 is a resistance heating element is inserted within the hydrogel and is configured to heat the hydrogel 300 in contact with the biomass. The hydrogel 300 in FIG. 3 is shown supported by a surface such as a membrane. In some embodiments, the biomass growth zone 300 is a hydrogel with immobilized biomass and immobilized resistance heating element 125. In some embodiments, the resistance heating element 125 is a resistance heating wire or carbon wire.

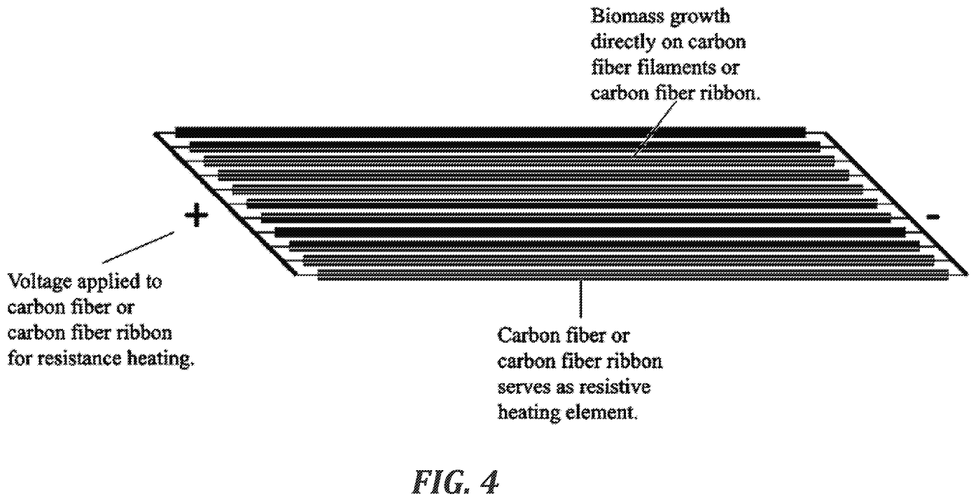

FIG. 4 shows biomass grown directly on carbon fiber resistance-heating elements, in accordance with the present technology. In certain embodiments, carbon fiber serves as both a resistance heating element 125 and directly as a support structure for biofilm growth. Biomass can adhere to and grow directly on carbon fiber filaments or carbon fiber ribbon. Such a device permits direct heating of the biofilm and creates a large surface area for biofilm growth due to the small filament diameter or ribbon thickness.

In other embodiments, the disclosure provides a biomass growth surface heated or cooled with gas convection or liquid convection. In certain embodiments, the gas and/or liquid are heated. In certain embodiments, the gas and/or liquid are cooled. In some embodiments, the gas comprises heated air or another heated composition of gas. In some embodiments, the gas comprises cooled air or another cooled composition of gas. In some embodiments, the liquid comprises heated water, heated wastewater, a heated glycol mixture, or combinations thereof. In certain embodiments, the liquid comprises cooled water, cooled wastewater, a cooled glycol mixture, or combinations thereof.

In some embodiments, heated gas or heated liquid supplies heat to the non-growth side of a biomass growth surface, to transfer heat to the biomass. For example, heated gas such as air could flow inside a hollow fiber membrane with biomass growing or deposited on the outside of the membrane, wherein the heated gas contacts the biomass growth surface. Such heated gas could comprise waste heat air and/or solar heated air. In certain embodiments, heated liquid such as waste heat liquid (e.g., water from a cooling operation, and/or glycol from a cooling operation) or solar heated liquid could be used to heat the biomass growth surface by passing the liquid over the surface opposite of where the biomass is grown, allowing the surface to conduct heat to the biomass from the liquid.

In some embodiments the temperature source 125 is a cooling source and is selected from a cold reservoir of air or water, liquid convection cooling, evaporative cooling, radiation cooling, and combinations thereof. In some embodiments, cooled gas or cooled liquid reduces the heat to the non-growth side of a biomass growth surface, to transfer heat away from the biomass. For example, cooled air, such as gas, may flow inside a hollow fiber membrane with the biomass is growing or deposited on the outside of the membrane, where the cooled gas contacts the biomass growth surface. Such cooled gas could comprise air cooled from a cold outdoor environment, cooled with a heat pump, cooled with a geothermal source, or cooled through heat exchange with a body of water or wastewater source. In some embodiments, liquid cooled from a cold outdoor environment, cooled with a heat pump, cooled with a geothermal source, or originating from a colder water body or wastewater source is configured to conduct heat away from the biomass growth surface by passing the liquid over the surface opposite of where the biomass is grown, allowing the surface to conduct heat away from the biomass and to the liquid.

The heat transfer in the foregoing embodiments can be controlled by monitoring the temperature of the biomass growth surface or biomass itself through infrared, thermocouple, or RTD temperature probes, and modifying the heat gain or heat loss by adjusting the flow rate of the heated or cooled gas or liquid, or adjusting the temperature of the gas or liquid, to maintain a specific biomass temperature. For example, the setpoint temperature range for a biological organism such as anammox could be between about 20° C. to about 60° C., from about 20° C. to about 50° C., from about 25° C. to about 45° C., or from about 30° C. to about 40° C. Such a temperature range permits a biological organism such as anammox, aerobic thermophilic organisms, and/or anaerobic thermophilic organisms to achieve their maximal growth rate, and consequently achieve high nitrogen-, carbon-, and/or phosphate removal or harvesting efficiencies.

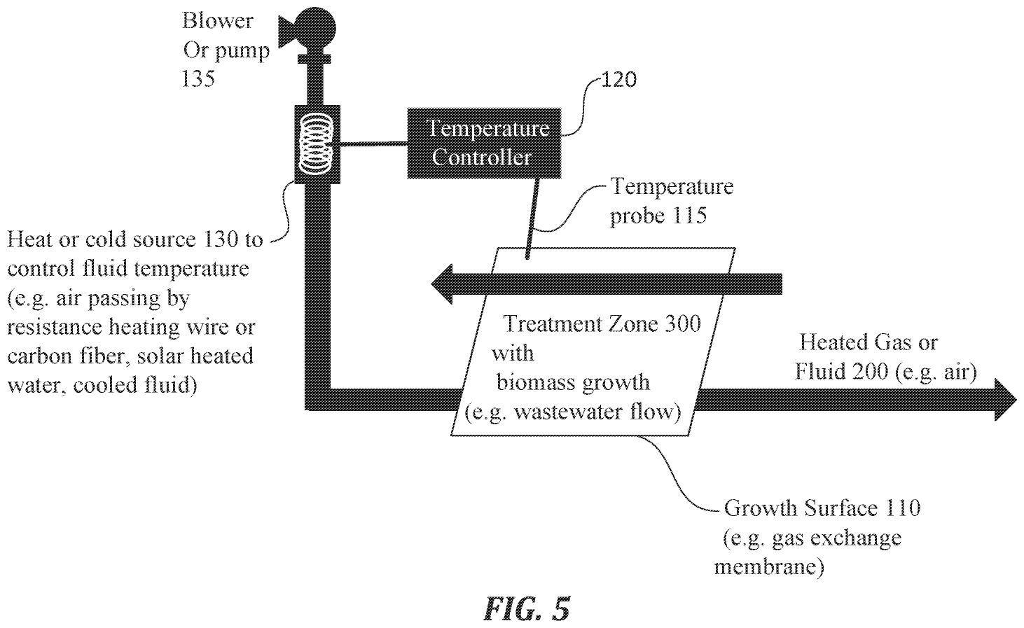

FIG. 5 shows a schematic for gas convection with the biomass growth on one membrane surface and the temperature-controlled gas in communication with the other membrane surface, in accordance with the present technology. In some embodiments, the system includes a blower or pump 135, a temperature controller 120, a temperature probe 115, and a growth surface 110, and a heated gas or fluid 200. In operation, the biomass growth surface is heated or cooled by a gas 200 (e.g., air) in communication with the biomass growth surface. In some embodiments, the biomass is configured to grow on the side of the growth surface 110, opposite of the heated or cooled side, and the growth surface is configured to conduct heat to the biomass or away from the biomass accordingly.

In some embodiments, a method for removing one or more of nitrogen, carbon, or phosphate from a medium, including contacting a temperature controlled biological growth surface 110 with the medium, wherein the biological growth surface 110 comprises an organism configured to remove the one or more of nitrogen, carbon, or phosphate from the medium when within a temperature range, and heating the temperature controlled biological growth surface 110 to within the temperature range in order to allow the organism to remove the one or more of nitrogen, carbon, or phosphate from the medium, or cooling the temperature controlled biological growth surface 110 to within the temperature range in order to allow the organism to remove the one or more of nitrogen, carbon, or phosphate from the medium is disclosed.

In some embodiments, a method for converting one or more of nitrogen, carbon, or phosphate from a medium, including contacting a temperature controlled biological growth surface 100 with the medium, wherein the biological growth surface comprises an organism configured to remove the one or more of nitrogen, carbon, or phosphate from the medium when within a temperature range, and heating the temperature controlled biological growth surface 110 to within the temperature range in order to allow the organism to convert the one or more of nitrogen, carbon, or phosphate from the medium, or cooling the temperature controlled biological growth surface 110 to within the temperature range in order to allow the organism to convert the one or more of nitrogen, carbon, or phosphate from the medium is disclosed. In some embodiments, the medium is a liquid. In some embodiments, the liquid comprises wastewater, contaminated water, water comprising the one or more of nitrogen, carbon, or phosphate, a carbon feed stock for fuel, a carbon feed stock for pharmaceutical production, a carbon feed stock for biochemical production, a carbon feed stock for monomer or polymer production, or a carbon feed stock for food production.

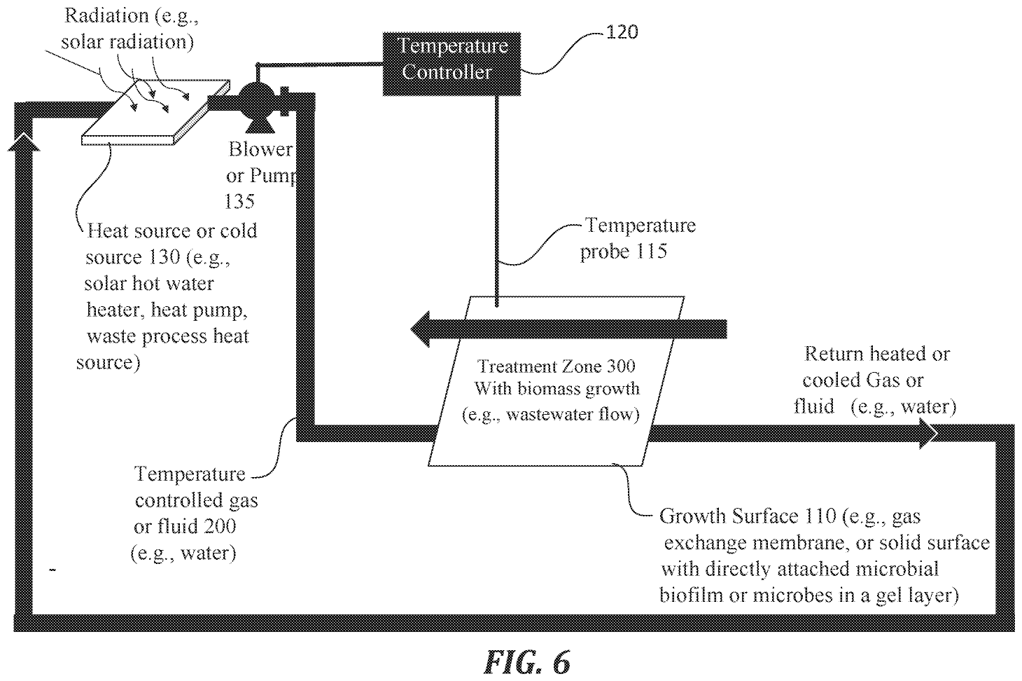

FIG. 6 shows a schematic for circulation of a temperature-controlled gas with the biomass growth on one membrane surface of the growth surface 110 and the liquid in communication with the other membrane surface of the growth surface 110, in accordance with the present technology. In FIG. 6, the biomass growth surface 110 is heated or cooled by a liquid 200 (e.g., solar-heated water) in communication with the growth surface 110. In operation, the biomass grows on the side opposite of the heated or cooled side, and the growth surface 110 conducts heat to the biomass or away from the biomass accordingly.

In other foregoing and following embodiments disclosed herein, the disclosure provides heating by radiation heat. In certain embodiments, radiation heat is in communication with the biomass directly, with the biomass growth surface, and/or a biomass growth surface such as a hydrogel or other porous matrix, which effects heating of the biomass, the biomass growth surface, and/or the hydrogel or other porous matrix. In certain embodiments, radiation heat comprises radiation in the visible range and/or infrared range. In other embodiments, radiation heat comprises a spectrum of radiation broader than the visible and infrared range. In further embodiments, radiation heat comprises a broad spectrum of radiation combined with a radiation filter which eliminates or reduces certain wavelengths of radiation from contacting the bacterial growth surface, such as certain wavelengths of UV radiation. Radiation heat comprises radiation emitted from a light and/or radiation emitted from a source such as the sun.

Heating the biological growth surface will take advantage of the properties of water. Water absorbs radiation heavily above 3000 nm, but the absorption of radiation by water quickly reduces at wavelengths below 3000 nm and has a minimum near 500 nm. In certain embodiments, radiation with a wavelength from around 200 nm to around 2500 nm is used to selectively heat suspended, biofilm-based, and/or hydrogel or other porous matrix in contact with a biomass, with minimal loss of energy to the surrounding water. The radiation can be provided electronically through lights at a specific wavelength or range of wavelengths, by solar radiation, or by filtered solar radiation. Filtered solar radiation permits specific wavelengths of light to penetrate the biological growth surface while concurrently preventing specific wavelengths which are harmful to biomass (e.g., a UV removing water filter) from contacting the biomass, biomass growth surface, and/or biomass growth surface such as a hydrogel or other porous matrix.

Absorption of radiation by the biomass can be enhanced by particle, pigment, or other additives added in proximity to organisms which have high absorption corresponding to the wavelength of radiation in which the device is exposed. Particles, pigments, or other additives comprise radiation-absorbing ability. For example, in embodiments in which a hydrogel or other porous matrix is in contact with a biomass, the embodiments can further comprise carbon black powder, wherein the carbon black powder can absorb radiation with a wavelength between about 200 nm to about 2,500 nm. Carbon black powder provides the further advantage that it is relatively inexpensive, chemically inert, and non-toxic to most organisms. In other embodiments, biomass which naturally absorb certain wavelengths of radiation can be paired with specific radiation at the wavelengths they absorb.

In further embodiments, a device can comprise more than one type of biomass, wherein each type of biomass has a different optimal temperature. In such embodiments, the concentration of particles or pigments added can be adjusted so that each type of biomass will experience different temperatures in near proximity to it or near each other. For example, nitrifying bacteria could be immobilized in hydrogel beads with no or low concentrations of radiation-absorbing particles, pigments, or other additives, while anammox bacteria could be immobilized in hydrogel beads with a high concentration of particles, pigments, or other additives. If both sets of hydrogel beads are placed in the same device and exposed to radiation (e.g., 500 nm green light) then nitrifiers will experience moderate temperatures while anammox bacteria will experience relatively moderate to high temperatures. This permits the device comprising anammox to operate with a wider range of biomass organisms and enables each type of biomass to exist at their maximum specific growth rate. In this example, anammox can receive its electron acceptor nitrite from the nitrifying organisms to then form dinitrogen gas leading to oxygen efficient nitrogen removal.

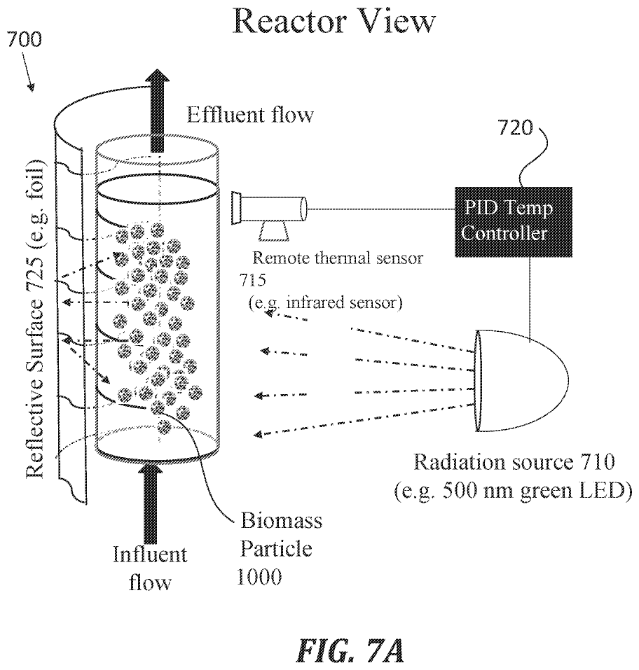

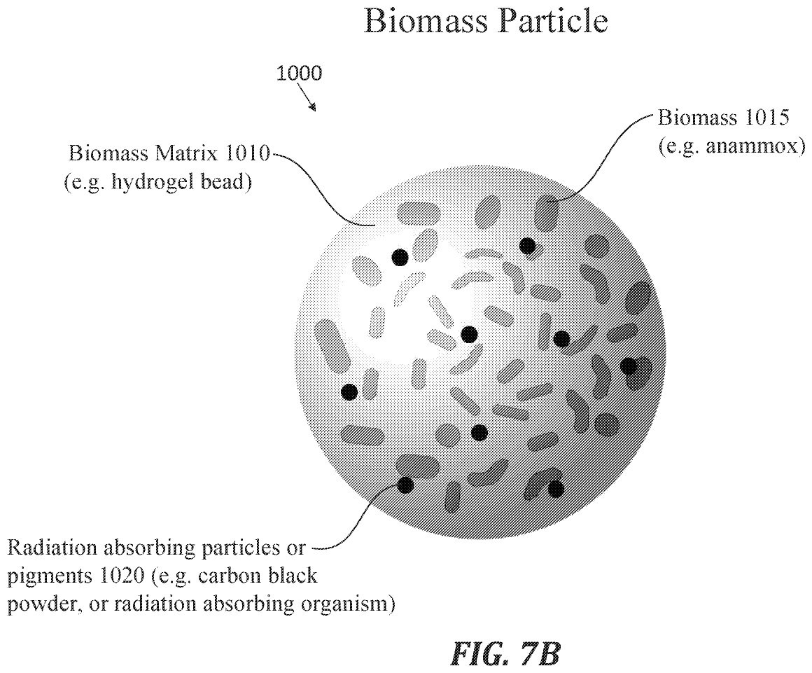

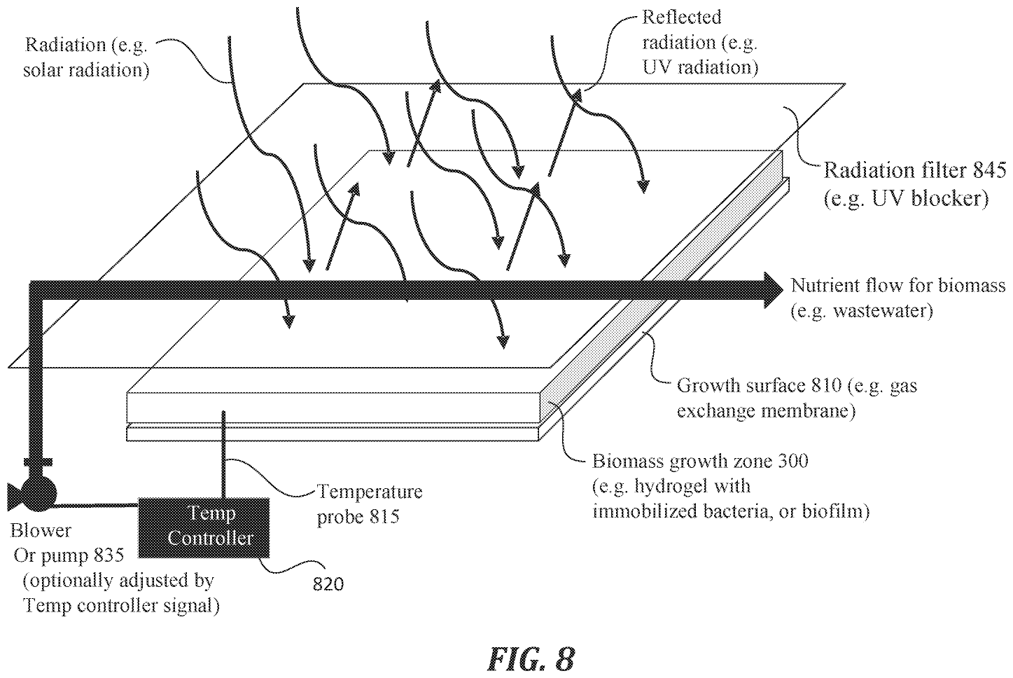

In certain embodiments, the device temperature is controlled by a control unit which measures the temperature of the biomass through an RTD, thermocouple, or infrared sensor, and controls the flux of radiation into the device based on the detected temperature of the biomass. For example, the setpoint temperature range for a biological organism such as anammox could be between about 20° C. to about 60° C., from about 20° C. to about 50° C., from about 25° C. to about 45° C., or from about 30° C. to about 40° C. Such a temperature range permits the biological organism such as anammox, methanogens, or yeast to achieve their maximal growth rate, and consequently achieve high nitrogen-, carbon-, and/or phosphate removal or harvesting efficiencies. The controller will reduce the flux of radiation from the radiation source (e.g., lights, LEDs, and/or solar) once the temperature of the biological organism such as anammox becomes higher than the setpoint and will increase the flux of radiation if the temperature falls lower than the setpoint. Otherwise, the flux of radiation will be kept at a proximally fixed level. The temperature will be sensed by, for example, an infrared sensor with a programmable logic controller that can distinguish the temperature signal of a liquid medium, such as water, from the biomass or hydrogel beads in contact with biomass. Additionally, temperature of the biomass can be controlled by changing the flow rate of nutrient fluid (gas or liquid) across the biomass. The concept of direct heating of biomass using radiation is shown in FIG. 7 and FIG. 8, described herein.

FIG. 7A shows direct heating of a biological growth surface 110 by radiation, in accordance with the present technology. In some embodiments, the biological growth surface 110 is incorporated into a reactor 700. In dome embodiments, the biological growth surface 110 is a plurality of beads, such as biomass particle 1000. The reactor 700 may further include a remote thermal sensor 715, such as an infrared sensor, a PID temperature controller 720, a radiation source 710, and a reflective surface 725. In some embodiments, the radiation source 710 is configured to heat the biomass particles 1000 with emitted light at 500 nm. In operation the hydrogel beads (or biomass particles, beads, etc.) 1000, in contact with biomass, are heated with green LED light (with a wavelength of about 500 nm) from the radiation source 710.

FIG. 7B shows an example biomass particle 1000, in accordance with the present technology. In some embodiments, a plurality of biomass beads 1000 are incorporated into a reactor, such as reactor 700. The biomass beads 1000 may include a biomass matrix 1010, such as a hydrogel bead, biomass 1015, such as organisms (e.g., anammox) and/or radiation absorbing particles 1020, pigments, or other additives (e.g., carbon black).

FIG. 8 shows direct heating of a biological growth surface by radiation, in accordance with the present technology. In some embodiments, the system includes a growth surface 810, a biomass growth zone 300, a temperature controller 820 connected to a temperature probe 815, a blower or pump 835, and a radiation filter 845. Specifically, FIG. 8 shows solar radiation and a radiation filter 845 configured to block certain wavelengths of light (e.g., UV, IR, or NIR) which can harm biomass growth. In some embodiments, the radiation filter is a UV blocker. The biomass growth zone 300 (e.g., a hydrogel in contact with biomass and/or including radiation-absorbing particles, pigments, or other additives) sits on a flat surface 810 for structural support (e.g., a gas exchange membrane). The temperature can be controlled by changing the flow rate of nutrient fluid (e.g., wastewater, feedstock, or contaminated water) through the reactor.

In other embodiments, the biomass growth surface is heated by its communication with radiation. This can be achieved with materials that absorb broad spectrum radiation, which shields the biomass from much of the direct radiation, while conducting heat to the biomass. The temperature of the biomass can be controlled by a radiation filter, which blocks or reflects certain wavelengths of radiation from reaching the biomass growth surface when the temperature gets too high. In such embodiments, the temperature can be controlled by adjusting the flowrate of nutrient flow across the biomass (e.g., flowrate of a wastewater stream), which in effect adjusts the rate of cooling. This technique is advantageous because the growth rate of many organisms is tied to temperature, and therefore the rate of nutrient supplied to organisms can be adjusted to meet higher or lower growth rates. Both nutrient supply and increased cooling can be achieved by increasing the flowrate of a nutrient rich liquid or gas across the biomass (e.g., increased rate of wastewater flow across the biomass).

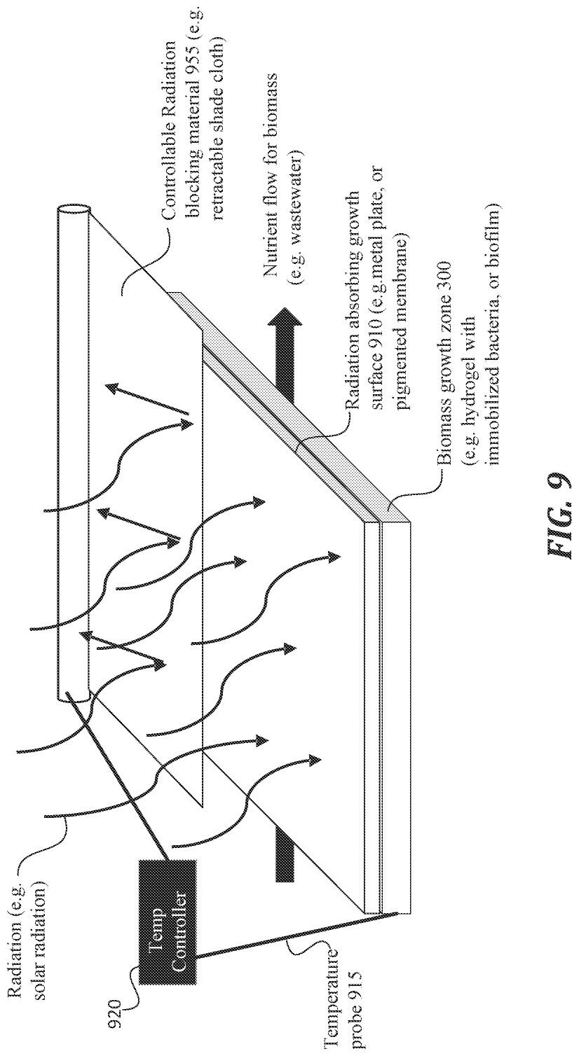

FIG. 9 shows an example of direct heating of the biomass growth surface by radiation, where the biomass growth surface 910 is configured to absorb solar radiation and to conduct heat to the biomass growth zone hydrogel 300 in contact with biomass, or to a biofilm, in accordance with the present disclosure. In some embodiments, the system further includes a temperature controller 920, a temperature probe 915, and a controllable radiation blocking material 955. In some embodiments, the radiation absorbing growth surface (or membrane) 910 supports the hydrogel matrix comprising the biomass (or biomass growth zone) 300, and the radiation-blocking filter (or material) 955 is used to decrease the intensity of radiation which communicates with the biomass growth surface membrane 910. Temperature of the biomass may be controlled by blocking part or all the radiation from the growth surface 910 when the temperature of the biomass gets too high or exceeds the set temperature range. In some embodiments, the radiation blocking material 955 is a cloth, shade cloth, or filter.

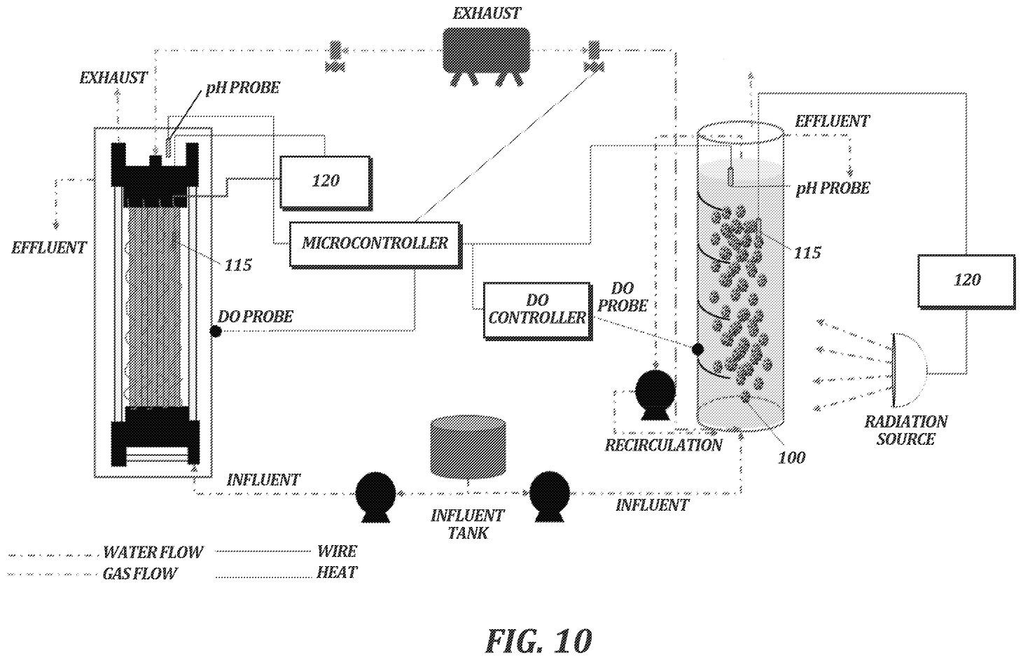

FIG. 10 shows a diagram of two reactor each having a biomass growth surface, in accordance with the present technology. In some embodiments, the biomass growth surface (such as biomass growth surface 110, 810, 910, or 1000) is incorporated as part of a reactor. In some embodiments, a system may include more than one reactor, each having a biomass growth surface as described herein. In some embodiments, each reactor may be heated or cooled using a different mechanism, such as a resistance heating wire and a radiation absorbing growth surface respectively. In some embodiments, each reactor may have the same type of biomass growth surface or temperature control. In some embodiments, the reactors share components, such as a microcontroller or temperature controller. In some embodiments, the reactor may include a microcontroller connected to a temperature controller and a pH probe. In some embodiments, the microcontroller is configured to direct the temperature controller and pH probe. In some embodiments, the reactor(s) are configured to control the pH, DO, and temperature by a systematic online system.

### Example

Two Anammox based reactors were tested at the pilot scale: a reactor system containing hydrogels embedded with Anammox biomass and carbon black (reactor 1: Hydrogel-on the left side of FIG. 10) and a heated membrane reactor system with Anammox biomass with Anammox biomass (reactor 2: Membrane Preload-on the right side of FIG. 10). The temperatures of biomass were controlled by using a novel infrared technology that heated the biomass in the beads (but not the water). In the other reactor, the temperature of biomass was controlled by heating the membrane (but not the water) with a temperature control system.

Schematic design of the reactors is shown in FIG. 10. Both reactors are designed to control the pH, DO, and temperature by a systematic online system. Both reactors were operated at 25° C. with a PID temperature controller and heating blocks (nichrome resistance heating for the membrane reactor and light radiation heating for the hydrogel reactor). This heating method is a novel way to only heat the biomass and not the water hence making these technologies of interest to cold mainstream applications.

According to the schematic design of the two reactors, major materials and equipment was purchased or prepared, and are summarized in Table 1.

TABLE 1
List of Major Equipment and Materials Prepared for Reactors

| ID | Item | Model | Usage |
|---|---|---|---|
| 1 | Temperature | Ink Bird ITC-106VH | To control temperature |
| | controller | | |
| 2 | Optical oxygen | PyroScience FSO2-C4 | To monitor DO |
| | meter | | |
| 3 | Air compressor | 3 Gallon ⅓ HP 100 PSI Oil- | To supply oxygen |
| | | Free Pancake Air Compressor | |
| 4 | pH Sensor | DFRobot Gravity | To monitor pH |
| 5 | microcontroller | ArDuino UNO R3 | Microcontroller to control |
| | | | temperature and DO |
| 6 | Realtime clock | Waiman DS1307 | Time clock for controller system |
| 7 | Peristaltic pump | Masterflex L/S ® Analog, | To control influent flow |
| 8 | Electric Solenoid | US solid USS2-00052 | To control air flow |
| | Valve | | |
| 9 | Power supply | Mh-120n-12 V | To provide power for membrane |
| | | | heating |
| 10 | Unistrut parts | Unistrut, bracket, board | To construct shelf in trailer for |
| | | | reactors |
| 11 | membrane | ZeeLung lab scale module | Functional part of membrane |
| | | | reactor |
| 12 | Polycarbonate | Transparent polycarbonate | Materials for reactor |
| | sheet/tubes | plastics | |

A site close to the primary clarifier at Everett wastewater treatment plant (WWTP) was selected for the pilot operation with consideration of access to electricity and reactor feeds (the effluent from the primary clarifier). A trailer located at the operation site was provided by the Everett Utility as the shelter for reactors. Connection of electricity and primary effluent to the trailer were set up by the staff of Everett staff. To improve the space utilization in the trailer, Unistrut shelves were made by the UW team members.

Before preparing hydrogel beads for the hydrogel reactor, a preliminary experiment was performed to investigate possible inhibition effect of carbon black, which is the radiation absorbent material, on the biomass activity and results showed no inhibition of radiation absorbent materials on biomass. Hydrogel beads were prepared by immobilizing Comammox and Anammox into 10% PVA-1% SA hydrogel beads and crosslinking with 2% CaCl2-3% boric acid followed by post-cure in 0.5 M Na₂SO₄. 0.125% homogenized carbon black powder were mixed into the hydrogel beads to make beads optically black to enhance their light absorbance capacity over a broad range of wavelengths.

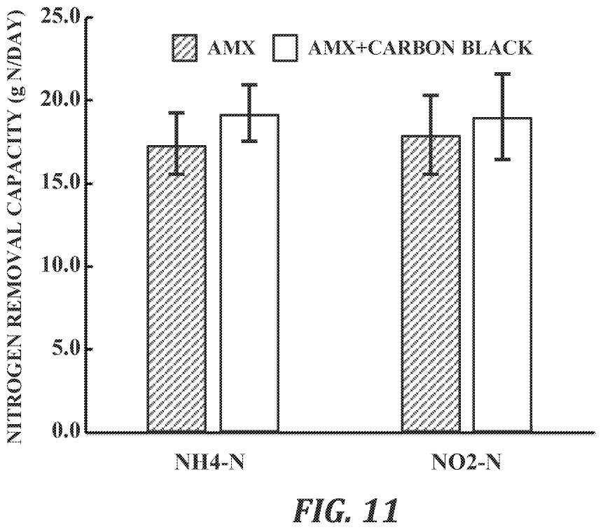

FIG. 11 is a graph showing the nitrogen removal capacity of annamox bacteria with and without carbon black, in accordance with the present technology. On the horizontal axis is the form of nitrogen being removed, NH₄—N and NO₂—N. On the vertical axis is the nitrogen removal capacity in g N/day. By adding carbon black to the annamox (AMX) bacteria, the nitrogen removal capacity of the annamox slightly improved for both forms of nitrogen, but not significantly (P>0.265).

A nichrome resistance heating module by the combination of nichrome wire and thermally conductive tape was constructed for the membrane reactor. A low voltage power box, which converts 120 V electricity to 5-24 V, was used for power supply for the heating module. The heating module was placed across and closely attached to the membrane fiber strips to ensure the direct heating of biomass on the membrane fiber strips. The heating module was tested before operation of reactors and showed sufficient capacity to heat the membrane to designed temperature.

For the heating module of the hydrogel reactor, a halogen lamp emitting light at wide spectrum was used as radiation source for heating in first attempt of the operation. This unsuitable radiation source promoted the growth of algae in the reactor and can be a reason of the failure of the first attempt of operation. An updated heating module was prepared with using LED lights which emitted light at a focused wavelength. This proposed new LED light source emitted light at a specific and human invisible wavelength of 900 nm which cannot be utilized by algae or be efficiently absorbed by water. A comparison of heating with water and water containing carbon black (light absorbent material used for hydrogel beads in the hydrogel reactor) demonstrated low absorbance by water and enhanced absorbance by addition of carbon black.

An Arduino was used for the microcontroller shown in FIG. 10. Code used for programming the microcontroller are as following:

| | |
|---|---|
| #define pHSensorPin1 0 | // the pH meter Analog output is connected |
| | with the Arduino's Analog |
| #define pHSensorPin2 1 | //assign the pin A0 and A1 to pH probes |
| unsigned long int avgValue1; | //Store the average value of the sensor feedback |
| unsigned long int avgValue2; | //Store the average value of the sensor feedback |

float b;
int buf[10],temp;

| | |
|---|---|
| #define DOSensor1 2 | //define the A2 as the input pin for sensor 1 |
| #define DOSensor2 3 | //define the A3 as the input pin for sensor 1 |
| int Solenoid1 = 2; | //Output pin2 for Solenoid Valve 1 |
| int Solenoid2 = 3; | //Output pin2 for Solenoid Valve 2 |

```
float Voltage1, DO1;
float Voltage2, DO2;
void setup( ) {
  // put your setup code here, to run once:
  Serial.begin(9600);//Sets baud rate for serial function
  Serial.print(“Ready”);
  Serial.println(“ “);
  pinMode(Solenoid1, OUTPUT);
  digitalWrite(Solenoid1, LOW);
  pinMode(Solenoid2, OUTPUT);
  digitalWrite(Solenoid2, LOW);
}
void loop( ) {
```

| | |
|---|---|
| for(int i=0;i<10;i++) | //Get 10 sample value from the sensor for smooth the value |

```
  {
  buf[i]=analogRead(pHSensorPin1);
  delay(10);
  }
```

| | |
|---|---|
| for(int i=0;i<9;i++) | //sort the analog from small to large |

```
  {
  for(int j=i+1;j<10;j++)
  {
    if(buf[i]>buf[j])
    {
    temp=buf[i];
    buf[i]=buf[j];
    buf[j]=temp;
    }
  }
  }
  avgValue1=0;
```

| | |
|---|---|
| for(int i=2;i<8;i++) | //take the average value of 6 center sample |

avgValue1+=buf[i];

| | |
|---|---|
| float phValue1=(float)avgValue1*5.0/1024/6; | //convert the analog into millivolt |
| phValue1=3.5*phValue1; | //convert the millivolt into pH value |

```
  Serial.print(“ pH1:”);
  Serial.print(phValue1,2);
```

| | |
|---|---|
| for(int i=0;i<10;i++) | //Get 10 sample value from the sensor for smooth the value |

```
  {
  buf[i]=analogRead(pHSensorPin2);
  delay(10);
  }
```

| | |
|---|---|
| for(int i=0;i<9;i++) | //sort the analog from small to large |

```
  {
  for(int j=i+1;j<10;j++)
  {
    if(buf[i]>buf[j])
    {
    temp=buf[i];
    buf[i]=buf[j];
    buf[j]=temp;
    }
  }
  }
  avgValue1=0;
```

| | |
|---|---|
| for(int i=2;i<8;i++) | //take the average value of 6 center sample |

avgValue2+=buf[i];

| | |
|---|---|
| float phValue2=(float)avgValue2*5.0/1024/6; | //convert the analog into millivolt |
| phValue1=3.5*phValue1; | //convert the millivolt into pH value |

```
  Serial.print(“ pH2:”);
  Serial.print(phValue2,2);
  Serial.println(“ “);
//rest for DO control
  Voltage1=analogRead(DOSensor1);
  DO1=Voltage1/45.5;
  Serial.print(“ DO1:”);
  Serial.print(DO1,2);// put your main code here, to run repeatedly:
  if (DO1 >= 0.30){ digitalWrite(Solenoid1,LOW);Serial.print(“; Aeration1 Off”);}
if (DO1 < 0.20){ digitalWrite(Solenoid1,HIGH);Serial.print(“; Aeration1 On”);
delay(3000);}
//rest for DO control2
  Voltage2=analogRead(DOSensor2);
  DO2=Voltage2/45.5;
  Serial.print(“ DO2:”);
  Serial.print(DO2,2);// put your main code here, to run repeatedly:
# if (DO2 >= 0.15){ digitalWrite(Solenoid2,LOW);Serial.print(“; Aeration2 Off”);}
# if (DO2 < 0.00){ digitalWrite(Solenoid2,HIGH);Serial.print(“; Aeration2 On”);
delay(3000);}
  Serial.println(“ “);
delay(3000);
}
```

Planed sampling and sample analysis for the two reactors are shown in Table 2. Routine sampling, sample analysis for physico-chemical parameters were performed by staff in the Everett WWTP. Additional samples of daily influent and effluent were stored at −20° C. for potential physico-chemical parameters and molecular analysis by UW team members. Reactor operational conditions, including flow, leakage of tubing, pressure of air or nitrogen tank, and temperature controls were monitored and maintained by the Everett plant staff and UW team members.

TABLE 2
A list of major analytes made with anaerobic
digester sludge to remove soluble chemical
oxygen demand (sCOD) in the membrane reactor

| Inventory | Parameter | Unit |
|---|---|---|
| physico- | Ammonia | mg/L-N |
| chemical | Nitrate | mg/L-N |
| | Nitrite | mg/L-N |
| | Ammonia | mg/L-N |
| | Nitrate | mg/L-N |
| | TSS | mg/L |
| | VSS | mg/L |
| | Soluble COD | mg/L |
| | pH | pH |
| | DO | mg/L-O² |
| | Temperature | ° C. |
| Molecular | qPCR-Universal 16S rRNA | gene copies/ng DNA |
| biology¹ | qPCR-Anammox 16S rRNA | gene copies/ng DNA |
| | qPCR-Comammox (amoA) | gene copies/ng DNA |
| | qPCR-AOB(amoA) | gene copies/ng DNA |
| | qPCR-NOB(nxrB) | gene copies/ng DNA |
| | qPCR-AOA (amoA) | gene copies/ng DNA |
| | 16S rRNA sequencing | microbial composition |

¹Sequencing conducted after all samples are collected

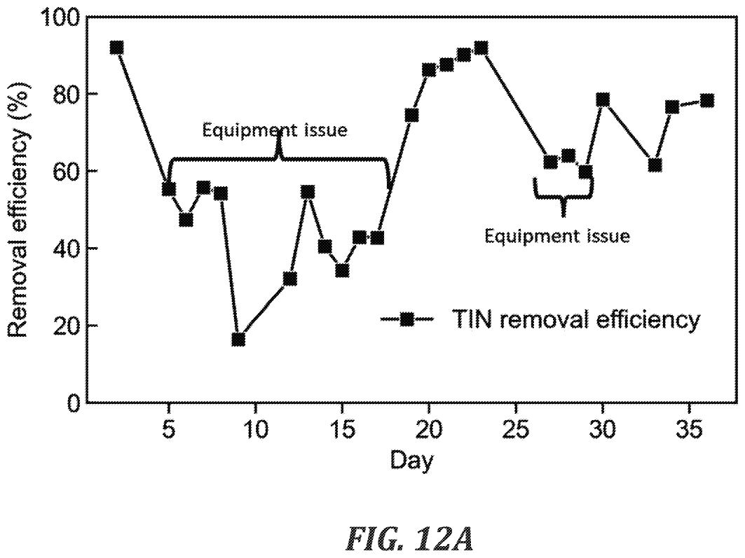

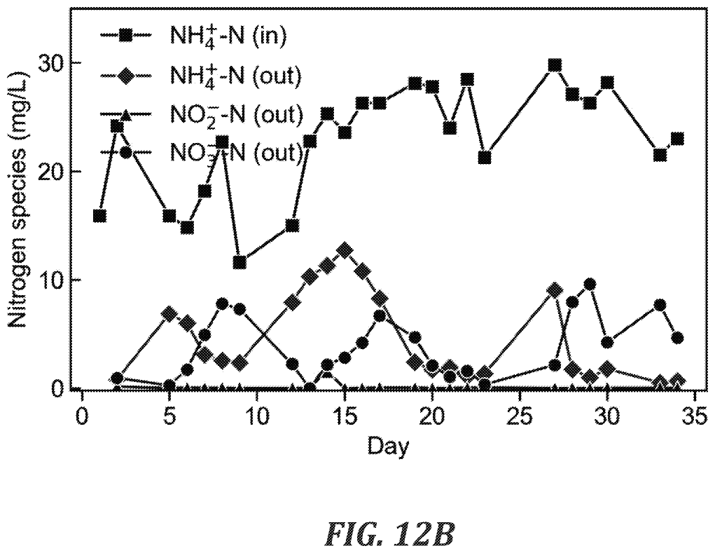

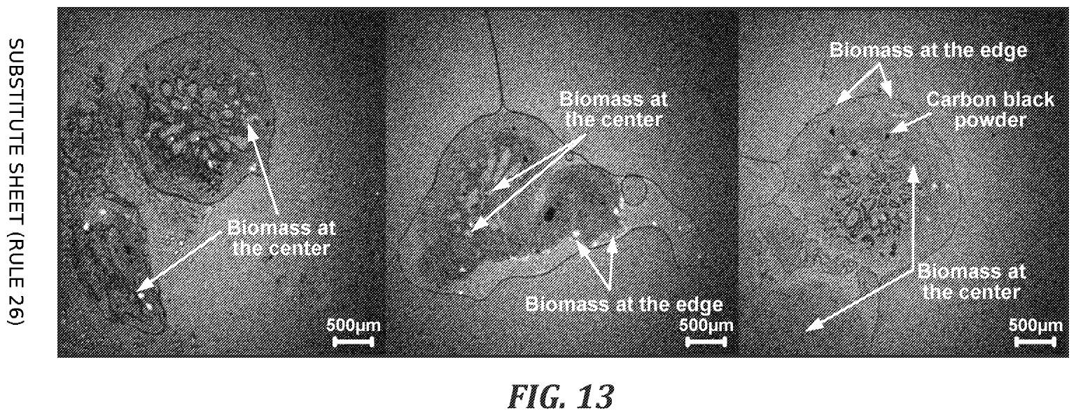

FIG. 12A is a graph showing the removal efficiency of total inorganic nitrogen (TIN), in accordance with the present technology. On the vertical axis is the removal efficiency in percentage. On the horizontal axis is the day. At the beginning of the pilot operation, the updated membrane reactor showed ideal nitrogen removal capacity with a removal efficiency of 96.7% and 92.1% for ammonia and TIN respectively, with a TIN concentration of 1.9 mg/L. However, sporadic disturbance to reactor operation happened in the following weeks due to incidental power outage, equipment issues with temperature control or aeration. The averaged removal efficiency was 94.7% and 82.2% for ammonia and TIN respectively, with an averaged TIN concentration of 4.4 mg/L in the effluent. During the week with no disturbance at all, the reactor demonstrated the capacity to reduce the TIN to lower than 3 mg/L in continuous days (around day 19-day 25), with an averaged TIN removal efficiency of 89.0% and an averaged effluent TIN concentration of 2.8%. The membrane reactor demonstrated very limited aeration requirement and highly efficient utilization of oxygen.

FIG. 12B is a graph showing the removal of various nitrogen species over time, in accordance with the present technology. On the vertical axis is the concentration of nitrogen species in mg/L. On the horizontal axis is the day. The plotted points show the concentration in the influent (in) and effluent (out) of the updated membrane reactor, of NH₄—N and NO₂—N. During the pilot operation of the updated membrane reactor, a minimal aeration rate at 2 cc/min was used to supply oxygen to the membrane and a DO at ˜ 0 mgO2/L were maintained in the bulk liquid in the reactor. This aeration is only 400% of the theoretical requirement for a complete nitrogen removal with influent NH₄—N concentration of 2 mM (28 mg/L), indicating an oxygen utilization efficiency of ˜25%. This aeration rate can be even lower after optimization as certain accumulation of nitrate resulting from the activity of nitride oxidizing bacteria (NOB) were still observed in the reactor suggesting that the oxygen supply had to be lowered.

The hydrogel reactor was setup at the Everett WWTP for pilot operation. FIG. 13 shows three microscopic images of biomass and carbon powder suspended in a biomass support, in accordance with the present technology. The growth of biomass within hydrogel beads was examined by hydrogel bead sectioning, SYBR Green staining, and microscope display. Growth of biomass within the hydrogel beads was confirmed as indicated by the green fluorescence (shown as bright white spots and labeled as such in FIG. 13) observed by microscope staining.

The hydrogel reactor was transferred to Everett WWTP for onsite pilot operation. A LED light source emitting light at a specific and human invisible wavelength of 900 nm was used as it cannot be utilized by algae, but that can still heat the biomass (but not the water).

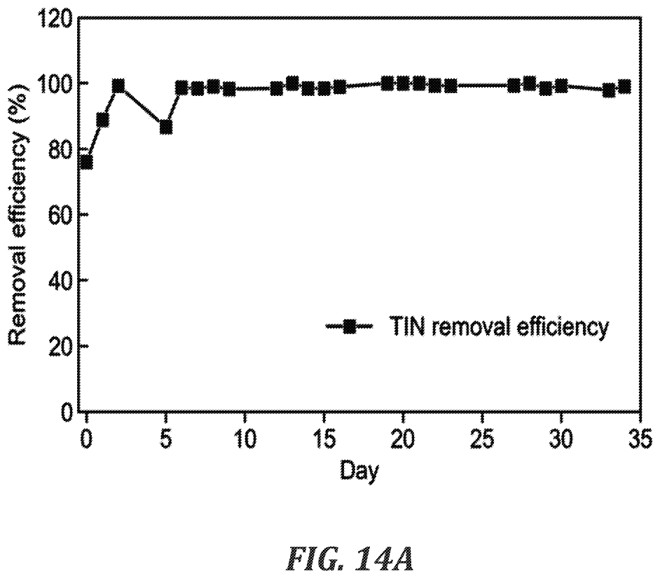

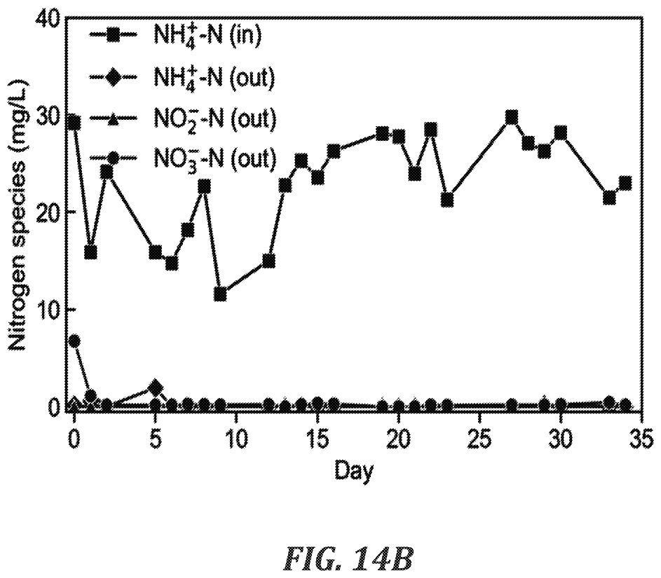

FIG. 14A is a graph showing the removal efficiency for TIN, in accordance with the present technology. On the vertical axis is the removal efficiency in percent. On the horizontal axis is the day. Plotted is the TIN removal efficiency. During pilot operation, the hydrogel reactor demonstrated excellent performance in nitrogen reduction. With an averaged ammonia concentration of 23.0 mg/L in the influent, the averaged ammonia and TIN removal efficiency of the hydrogel reactor was 99.1% and 98.1% respectively during the entire pilot operation. The ammonia and TIN concentrations in the effluent were nearly undetectable, with an averaged concentration of 0.13 mg/L and 0.37 mg/L respectively.

FIG. 14B is a graph showing the removal of various nitrogen species over time, in accordance with the present technology. On the vertical axis is the nitrogen species concentration in mg/L. On the horizontal axis is the day. Plotted are the concentrations of NH₄—N and NO₂—N in both influent (in) and effluent (out). As shown, NH₄—N was present in the influent water, but levels of NH₄—N in effluent were negligible. No unexpected activity of NOB was observed in the reactor as indicated by the negligible accumulation of nitrate.

1. A temperature controlled biological growth surface, comprising:
a biomass growth surface configured to support a biomass; and
a temperature source in thermal communication with the biomass growth surface.

2. The temperature controlled biological growth surface of claim 1, further comprising a biomass in contact with the biomass growth surface.

3. The temperature controlled biological growth surface of claim 2, wherein the biomass comprises an organism or a consortium of organisms selected from anaerobic ammonium oxidizing (anammox) bacteria, nitrifying bacteria, methanogen or methanotroph bacteria, denitrifying organisms, sulfate reducers, sulfate oxidizers, carbon- or nitrogen-processing heterotrophic or phototrophic prokaryotes, carbon- or nitrogen-processing heterotrophic or phototrophic eukaryotes, fungal strains, yeast, and combinations thereof.

4. The temperature controlled biological growth surface of claim 1, wherein the biomass growth surface is a membrane.

5. The temperature controlled biological growth surface of claim 4, wherein the membrane is selected from a gas-exchanging membrane, a ceramic membrane, a metallic membrane, a hollow-fiber membrane, or a combination thereof.

6. The temperature controlled biological growth surface of claim 1, wherein the biomass growth surface has a form factor selected from substantially planar, substantially cylindrical, and a plurality of beads.

7. The temperature controlled biological growth surface of claim 1, wherein the biological growth surface comprises a carbon fiber filament, a carbon fiber ribbon, a metal plate or tube, a metallic surface modified to accept a biofilm or hydrogel coating, a pigmented membrane, or a combination thereof.

8. The temperature controlled biological growth surface of claim 1, wherein the temperature source is a heat source selected from resistive heat, gas convention heat, liquid convection heat, radiation heat, heat from combustion, heat from friction, and combinations thereof.

9. The temperature controlled biological growth surface of claim 8, wherein the heat source operates at least partially by radiation heat and wherein a radiation includes a peak wavelength in the visible or infrared regions.

10. The heated biological growth surface of claim 9, wherein the biomass further comprises a particle, a pigment, other additives, or a combination thereof configured to absorb the radiation.

11. The temperature controlled biological growth surface of claim 1, wherein the temperature source is a cooling source selected from a heat pump, a cold reservoir of air or water, liquid convection cooling, evaporative cooling, radiation cooling, and combinations thereof.

12. The temperature controlled biological growth surface of claim 1, wherein the temperature of the biological growth surface is controlled by a control unit.

13. The temperature controlled biological growth surface of claim 12, wherein the control unit is selected from one or more of a temperature sensor, a radiation filter, a radiation intensity control device, a biomass nutrient fluid rate control device, or a combination thereof.

14. The temperature controlled biological growth surface of claim 12, wherein the control unit comprises a proportional-integral-derivative (PID) temperature controller and a temperature sensor, wherein the temperature sensor is an infrared sensor, a thermocouple, or a resistance temperature detector (RTD).

15. The temperature controlled biological growth surface of claim 1, wherein the biomass growth surface is configured to support a biomass comprising a hydrogel or other porous matrix.

16. The temperature controlled biological growth surface of claim 15, wherein the hydrogel or other porous matrix is configured to be in contact with the biomass.

17. A method for removing one or more of nitrogen, carbon, or phosphate from a medium, comprising:
contacting a temperature controlled biological growth surface according to claim 1 with the medium, wherein the biological growth surface comprises an organism configured to remove the one or more of nitrogen, carbon, or phosphate from the medium when within a temperature range; and
heating the temperature controlled biological growth surface to within the temperature range in order to allow the organism to remove the one or more of nitrogen, carbon, or phosphate from the medium; or
cooling the temperature controlled biological growth surface to within the temperature range in order to allow the organism to remove the one or more of nitrogen, carbon, or phosphate from the medium.

18. A method for converting one or more of nitrogen, carbon, or phosphate from a medium, comprising:
contacting a temperature controlled biological growth surface according to claim 1 with the medium, wherein the biological growth surface comprises an organism configured to remove the one or more of nitrogen, carbon, or phosphate from the medium when within a temperature range; and
heating the temperature controlled biological growth surface to within the temperature range in order to allow the organism to convert the one or more of nitrogen, carbon, or phosphate from the medium; or
cooling the temperature controlled biological growth surface to within the temperature range in order to allow the organism to convert the one or more of nitrogen, carbon, or phosphate from the medium.

19. The method of claim 17, wherein the medium is a liquid.

20. The method of claim 19, wherein the liquid comprises wastewater, contaminated water, water comprising the one or more of nitrogen, carbon, or phosphate, a carbon feed stock for fuel, a carbon feed stock for pharmaceutical production, a carbon feed stock for biochemical production, a carbon feed stock for monomer or polymer production, or a carbon feed stock for food production.
